SEQUENCE LISTING (18 sequences)

SEQ ID NO: 1 - DNA, 20 bp, Artificial Sequence
  Primer for OCT1 - Forward
aagcacaccgtcatcctgat

SEQ ID NO: 2 - DNA, 20 bp, Artificial Sequence
  Primer for OCT1 - Reverse
ccaggttctctgcttcttca

SEQ ID NO: 3 - DNA, 20 bp, Artificial Sequence
  Primer for OCT2 - Forward
gccagtgcatgaggtatgaa

SEQ ID NO: 4 - DNA, 20 bp, Artificial Sequence
  Primer for OCT2 - Reverse
agttgggaatcacataggcc

SEQ ID NO: 5 - DNA, 20 bp, Artificial Sequence
  Primer for MATE1 - Forward
atacgggagccagaacttga

SEQ ID NO: 6 - DNA, 20 bp, Artificial Sequence
  Primer for MATE1 - Reverse
gttgacaaggttagcagcga

SEQ ID NO: 7 - DNA, 20 bp, Artificial Sequence
  Primer for CTR1 - Forward
tcatcatcctgagccttgtc

SEQ ID NO: 8 - DNA, 20 bp, Artificial Sequence
  Primer for CTR1 - Reverse
gactggatgagatgtcccta

SEQ ID NO: 9 - DNA, 20 bp, Artificial Sequence
  Primer for CTR2 - Forward
gtatgagggcatcaaggttg

SEQ ID NO: 10 - DNA, 20 bp, Artificial Sequence
  Primer for CTR2 - Reverse
cttcatctcagtcaccagga

SEQ ID NO: 11 - DNA, 20 bp, Artificial Sequence
  Primer for MRP1 - Forward
aacattgctacaaggcggtg

SEQ ID NO: 12 - DNA, 20 bp, Artificial Sequence
  Primer for MRP1 - Reverse
ctttgactccttccctaaac

SEQ ID NO: 13 - DNA, 20 bp, Artificial Sequence
  Primer for MRP2 - Forward
acttggtcgtcttctgttcc

SEQ ID NO: 14 - DNA, 20 bp, Artificial Sequence
  Primer for MRP2 - Reverse
gaggcaacatctatcccatc

SEQ ID NO: 15 - DNA, 20 bp, Artificial Sequence
  Primer for ATP7A - Forward
agcagcagattgggaaagtg

SEQ ID NO: 16 - DNA, 20 bp, Artificial Sequence
  Primer for ATP7A - Reverse
cagtggagcagtaacagtca

SEQ ID NO: 17 - DNA, 20 bp, Artificial Sequence
  Primer for ATP7B - Forward
tcatcctggtggttgccata

SEQ ID NO: 18 - DNA, 20 bp, Artificial Sequence
  Primer for ATP7B - Reverse
cttgatgatgtcacctcgct

(19) United States
(12) Patent Application Publication
(10) Pub. No.: US 2015/0087677 A1
(43) Pub. Date: Mar. 26, 2015

(54) METHODS FOR TREATING VESTIBULOTOXICTY

(71) Applicant: Sensorion, Clapiers (FR)

(72) Inventor: Sophie Gaboyard-Niay, Gigean (FR)

(21) Appl. No.: 14/555,997

(22) Filed: Nov. 28, 2014

Related U.S. Application Data
(63) Continuation-in-part of application No. PCT/EP2013/061205, filed on May 30, 2013.
(60) Provisional application No. 61/653,074, filed on May 30, 2012.

(30) Foreign Application Priority Data
Nov. 29, 2012 (EP) 12194915.0

(51) Int. Cl. A61K 31/4439; A61K 31/341; A61K 31/4174

(52) U.S. Cl. CPC A61K 31/4439; A61K 31/4174; A61K 31/341
USPC 514/338; 514/400; 514/461

(57) ABSTRACT

The present invention relates to a method for treating platinum drugs induced vestibulotoxicity, comprising administering a compound that impairs or prevents accumulation of platinum drugs within vestibular hair cells and/or vestibular primary neurons.

### CROSS REFERENCE TO RELATED APPLICATIONS

The present application is a Continuation-in-part of International Patent Application No. PCT/EP2013/061205 filed May 30, 2013, which claims the benefit of U.S. Provisional Application No. 61/653,074 filed May 30, 2012, and claims priority to European Patent Application No. 12194915.0 filed on Nov. 29, 2012, the entire contents of which are incorporated herein by reference in their entireties for all purposes.

### FIELD OF INVENTION

The present invention relates to methods for treating platinum drugs induced vestibulotoxicity.

### BACKGROUND OF INVENTION

The inner ear is the innermost part of the vertebrate ear where two sensory organs are hosted in the temporal bone:
The cochlea, dedicated to the auditory function, converting sound pressure patterns from the outer ear into electrochemical impulses which are passed on to the brain via the auditory nerve. The cochlea constitutes the ventral region of the inner ear and it contains the organ of Corti that comprises mechanosensory hair cells and supporting cells.
The vestibular system, dedicated to the balance function, through acceleration, and gravity sensing. This organ has a totally different function from cochlea, said function consisting in the detection of linear and angular accelerations of the head in order to transmit to the brain information on movements to achieve the equilibration function in collaboration with visual and proprioceptive information. The vestibular system is the dorsal region of the inner ear where the semicircular canals converge, close to the cochlea. The vestibular system contains the sensory epithelia named: anterior crista, horizontal crista, posterior crista, utricle, and saccule. It is also constituted of mechanosensory hair cells that convert mechanical actions into electrical potentials, and supporting cells.

Although these two organs are constituted of the same type of cells, their distinct morphology and function imply differences in molecular and cellular mechanisms underlying their action. The vestibular system and the cochlea exhibit organ-dependent differences. Indeed, some receptors or proteins expressed in both organs will enhance different physiological mechanisms in both organs. For example, neurotrophin factors have totally differential expression during development and in adult mammals with NT3 preferentially expressed in the cochlea and BDNF mostly segregated to the vestibule (for review see Fritsch et al., 2004).

Deficiencies in BDNF and/or NT3 such as null mutant will affect the cells of the vestibular system essentially when BDNF is lacking while the effects on the cells of the cochlea are not really noticeable until NT3 expression is impaired (Fritzsch B. et al. 2004 Prog. Brain Res. 146: 265-278).

In another example, some pathological mutations (e.g. TMPRSS3 resulting in DFNB8/10) induce severe and early deafness in children but only result in a late and mild loss of vestibular function in adults (Fasquelle et al., 2011).

As another example of differences between cochlea and vestibule, aminoglycosides are also well described to be preferentially toxic to either cochlea or vestibule: Amikacin, neomycin and dihydrostreptomycin tend to be more cochleotoxic while gentamicin and streptomycin are more likely to target the vestibular sensory epithelium (for review see Xie et al., 2011).

In summary, such structural, functional, developmental or pathological differences between the cochlea and the vestibule lead the search for vestibular therapeutic treatment as being highly specific to this peculiar sensory organ.

Platinum drugs are among the most active anticancer agents and are used as single agent or in combination with other cytotoxic agents and/or radiation therapy in the management of a broad spectrum of human malignancies. Platinum derivative vestibulotoxicity or vestibular toxicity, leads to impaired vestibular/balance function. Schaeffer et al. (Cancer 1981, 47:857-859) described the first human case of vestibular toxicity following cisplatin therapy.

Therefore the present invention aims at providing compounds for treating specifically vestibulotoxicity induced by platinum drugs.

### SUMMARY

One object of the invention is a compound that impairs or prevents accumulation of platinum drugs within vestibular hair cells and/or vestibular primary neurons for use in treating platinum drugs induced vestibulotoxicity.

In one embodiment, said compound promote the protection, survival or regeneration of the vestibular hair cells and/or vestibular primary neurons.

In another embodiment, the platinum drugs are cisplatin, carboplatin, oxaliplatin, satraplatin, picoplatin, tetraplatin, transplatin, nedaplatin, ormaplatin, PtCl2[R,R-DACH], pyriplatin, ZD0473, BBR3464 or Pt-1C3.

In another embodiment, said compound modulates platinum drugs flux through vestibular hair cells and/or vestibular primary neurons, or impairs or reduces the influx or uptake of platinum drugs in vestibular cells and/or vestibular primary neurons, or impairs or increases the efflux of platinum drugs in the vestibular hair cells and/or vestibular primary neurons.

In another embodiment, said compound is an OCT (organic cation transporter) inhibitor, a CTR (copper transporters) inhibitor, a MATE (multidrug and toxin extrusion) transporters activator, a MRP (multidrug resistance associated protein) transporters activator, or an ATP7A or ATP7B transporters activator.

In another embodiment, said compound is cimetidine, linagliptin, agmatine, chlorpromazine, doxepin, erlotinib, gefitinib, metformin, amitriptyline, carvedilol, chloroquine, clonidine, desloratadine, diphenhydramine, disopyramide, fenfluramine, flecainide, flurazepam, imipramine, ipratropiumbromide, levomethadone, mefloquine, mexiletine, propafenone, propranolol, quinidine, sibutramine, tamoxifen, trimethoprim, verapamil, imatinib, and 6β-hydroxycortisol, collagen/β1 integrin, lipopolysaccharide (LPS), copper sulphate, 4-phenylbutyrate (4-PBA), curcumin, nizatidine, ranitidine, pantoprazole, rabeprazole, lansoprazole or omeprazole.

Another object of the invention is a pharmaceutical composition or medicament comprising a compound that impairs or prevents accumulation of platinum drugs within vestibular hair cells and/or vestibular primary neurons as described here above for use in treating platinum drugs induced vestibulotoxicity.

Another object of the invention is a method for treating platinum drugs induced vestibulotoxicity, comprising administering a compound that impairs or prevents accumulation of platinum drugs within vestibular hair cells and/or vestibular primary neurons.

In one embodiment, said compound promote the protection, survival or regeneration of the vestibular hair cells and/or vestibular primary neurons.

In one embodiment, the platinum drugs are cisplatin, carboplatin, oxaliplatin, satraplatin, picoplatin, tetraplatin, transplatin, nedaplatin, ormaplatin, PtCl2[R,R-DACH], pyriplatin, ZD0473, BBR3464 or Pt-1C3.

In one embodiment, said compound modulates platinum drugs flux through vestibular hair cells and/or vestibular primary neurons, or impairs or reduces the influx or uptake of platinum drugs in vestibular cells and/or vestibular primary neurons, or impairs or increases the efflux of platinum drugs in the vestibular hair cells and/or vestibular primary neurons.

In one embodiment, said compound is an OCT (organic cations transporter) inhibitor, a CTR (copper transporters) inhibitor, a MATE (multidrug and toxin extrusion) transporters activator, a MRP (multidrug resistance associated protein) transporters activator, or an ATP7A or ATP7B transporters activator.

In one embodiment, said compound is cimetidine, linagliptin, amantadine, memantine, agmatine, chlorpromazine, doxepin, erlotinib, gefitinib, metformin, amitriptyline, carvedilol, chloroquine, clonidine, desloratadine, diphenhydramine, disopyramide, fenfluramine, flecainide, flurazepam, imipramine, ipratropiumbromide, levomethadone, mefloquine, mexiletine, propafenone, propranolol, quinidine, sibutramine, tamoxifen, trimethoprim, verapamil, imatinib, and 6β-hydroxycortisol, collagen/β1 integrin, lipopolysaccharide (LPS), copper sulphate, 4-phenylbutyrate (4-PBA), curcumin, nizatidine, ranitidine, pantoprazole, rabeprazole, lansoprazole or omeprazole.

Preferably, said compound is cimetidine, nizatidine, ranitidine, pantoprazole, rabeprazole, lansoprazole or omeprazole.

### DETAILED DESCRIPTION

The present invention relates to a method for treating platinum drugs induced vestibulotoxicity in a subject in need thereof, comprising the administration to the subject of a therapeutically effective amount of a compound that impairs/prevents accumulation of platinum drugs within vestibular hair cells and/or vestibular primary neurons.

As used herein, the terms “treating” or “treatment” refers to both therapeutic treatment and prophylactic or preventative measures; wherein the object is to prevent or slow down (lessen) platinum drugs induced vestibulotoxicity. Those in need of treatment include those already with platinum drugs induced vestibulotoxicity as well as those prone to have platinum drugs induced vestibulotoxicity or those in whom platinum drugs induced vestibulotoxicity is to be prevented. A subject or mammal is successfully “treated” for platinum drugs induced vestibulotoxicity if, after receiving a therapeutic amount of a compound according to the methods of the present invention, the patient shows observable and/or measurable reduction in or absence of one or more of the following: reduction in the number of pathogenic cells; reduction in the percent of total cells that are pathogenic; and/or relief to some extent, of one or more of the symptoms associated with platinum drugs induced vestibulotoxicity; and improvement in quality of life issues. The above parameters for assessing successful treatment and improvement in platinum drugs induced vestibulotoxicity are readily measurable by routine procedures familiar to a physician.

Another object of the invention is a compound that impairs/prevents accumulation of platinum drugs within vestibular hair cells and/or vestibular primary neurons for treating or for use in treating platinum drugs induced vestibulotoxicity.

As used herein, vestibulotoxicity refers to impairment of the vestibule function leading to vestibular deficits. Said impairment may be due to vestibular hair cells, preferably to the vestibular hair cells platinum drugs induced damages, insults or loss, and/or platinum drugs induced lesions in the vestibule.

Means for evaluating vestibulotoxicity include, but are not limited to, vertical subjective evaluation, vestibulonystagmography (VNG), vestibulo oculomotor reflex (VOR) evaluation, myogenic reflex and Head Impulse Test (HIT).

As used herein, the term “treatment” refers to preventing (i.e. keeping from happening), reducing or alleviating at least one adverse effect or symptom of a disease, disorder or condition associated with a deficiency in or absence of an organ, tissue or cell function. Accordingly the aim of the invention is to provide an ending of the vestibulotoxicity or an amelioration of the subject's condition by protecting or restoring the functionality or part of the functionality of the vestibular endorgans. The invention also aims at preventing any lesion to appear or preventing lesions already present to increase.

The present invention thus provides a method for treating, protecting, restoring the vestibular neuronal network or the vestibular functionality or preventing vestibulotoxicity in a subject treated or going to be treated with platinum drugs. The compounds of the invention may be administrated to promote the protection, survival or regeneration of the vestibular hair cells and/or vestibular primary neurons.

As used herein, the term “subject” denotes a mammal, such as a rodent, a feline, a canine, and a primate. Preferably, a subject according to the invention is a human.

As used herein, platinum drugs refer to platinum based drugs such as cisplatin (cis-diamminedichloroplatinum(II)), carboplatin (cis-diammine-1,1-cyclobutane-dicarboxylatoplatinum(II)), oxaliplatin (trans-L-diaminocyclohexane-oxalotoplatinum(II)), satraplatin (JM216 (bis-acetato-amminedichloro-cyclohexylamineplatinum(IV) and JM118(cis-amminedichloro-cyclohexylamineplatinum(II)), picoplatin (cis-amminedichloro,2-methylpyridineplatinum(II)), tetraplatin (cis-diamminetetrachloroplatinum(IV)), transplatin (trans-diamminedichloroplatinum(II)), nedaplatin (cis-diammine-glycolatoplatinum(II)), ormaplatin (trans-L-1,2-diaminocyclohexane-tetrachloroplatinum(IV)), PtCl2[R,R-DACH] (1,2-diamminecyclohexane-dichloroplatinum(II)), pyriplatin (cis-diammine(pyridine)-chloroplatinum(II)), ZD0473, BBR3464, Pt-1C3.

In one embodiment of the invention, said compound that impairs accumulation of platinum drugs within vestibular hair cells, is a compound that reduces accumulation of platinum drugs within vestibular hair cells and/or vestibular primary neurons.

An example of method for determining whether a compound impairs accumulation of platinum drugs within hair cells is the following:

Organotypic 3D cultures of vestibular explants are incubated with lethal dose of cisplatin for 48 hrs. Hair cell loss induced by cisplatin toxicity is evaluated using immunohistochemistry and morphometry as quantitative analysis. Hair cell body is stained using labeling of MyosinVIIA expression, while hair bundles expressing actin are labeled using fluorescent conjugated phalloidin. Apoptotic cells is evaluated using the tunnel method. Number of hair cells, hair bundles and apoptotic cells are quantified.

In another embodiment of the invention, said compound modulates platinum drugs flux through vestibular hair cells and/or vestibular primary neurons.

In another embodiment of the invention, said compound impairs or reduces the influx or uptake of platinum drugs in the vestibular hair cells and/or vestibular primary neurons.

In another embodiment of the invention, said compound impairs or increases the efflux of platinum drugs from the vestibular hair cells and/or vestibular primary neurons.

In one embodiment of the invention, said compound is an inhibitor of OCT (organic cation transporters)-mediated uptake of platinum drugs in the vestibular hair cells and/or vestibular primary neurons.

In one embodiment of the invention, said OCT inhibitor is an inhibitor of OCT1, OCT2 and/or OCT3, encoded by SLC22A1, SLC22A2 and SLC22A3 respectively. Preferably, said OCT inhibitor is an OCT2 inhibitor.

As used herein, the term “inhibitor of OCT-mediated uptake of platinum drugs” refers to any compound that inhibits, impairs or prevents the transport of platinum drugs by hair cells and/or vestibular primary neurons. For example, said compound may be an inhibitor of the transporter function of the OCT. Another example is a compound that is a substrate of OCT, which means that the compound interacts with the OCT and enters in competition with the platinum drugs, thereby reducing or inhibiting the uptake of platinum drugs within vestibular hair cells and/or vestibular primary neurons.

Examples of inhibitors of OCT-mediated uptake of platinum drugs in the hair cells include, but are not limited to, cimetidine, linagliptin, agmatine, chlorpromazine, doxepin, erlotinib, gefitinib, metformin, amitriptyline, carvedilol, chloroquine, clonidine, desloratadine, diphenhydramine, disopyramide, fenfluramine, flecainide, flurazepam, imipramine, ipratropiumbromide, levomethadone, mefloquine, mexiletine, propafenone, propranolol, quinidine, sibutramine, tamoxifen, trimethoprim, verapamil, imatinib, 6β-hydroxycortisol, amantadine, memantine, nizatidine, ranitidine, pantoprazole, rabeprazole, lansoprazole and omeprazole. In one embodiment of the invention, said inhibitors of OCT-mediated uptake of platinum drugs in the vestibular hair cells is not cimetidine.

In one embodiment of the invention, said inhibitor of OCT-mediated uptake of platinum drugs in the vestibular hair cells is selected from the group consisting of cimetidine, nizatidine, ranitidine, pantoprazole, rabeprazole, lansoprazole and omeprazole.

In one embodiment, said inhibitor of OCT-mediated uptake of platinum drugs in the vestibular hair cells is cimetidine.

In one embodiment, said inhibitor of OCT-mediated uptake of platinum drugs in the vestibular hair cells is nizatidine.

In one embodiment, said inhibitor of OCT-mediated uptake of platinum drugs in the vestibular hair cells is ranitidine.

In one embodiment, said inhibitor of OCT-mediated uptake of platinum drugs in the vestibular hair cells is pantoprazole.

In one embodiment, said inhibitor of OCT-mediated uptake of platinum drugs in the vestibular hair cells is rabeprazole.

In one embodiment, said inhibitor of OCT-mediated uptake of platinum drugs in the vestibular hair cells is lansoprazole.

In one embodiment, said inhibitor of OCT-mediated uptake of platinum drugs in the vestibular hair cells is omeprazole.

In one embodiment of the invention, said inhibitors of OCT-mediated uptake of platinum drugs in the vestibular hair cells is amantadine.

In one embodiment of the invention, said inhibitors of OCT-mediated uptake of platinum drugs in the vestibular hair cells is memantine.

In one embodiment of the invention, said inhibitors of OCT-mediated uptake of platinum drugs in the vestibular hair cells is linagliptin.

Example of a method for determining whether a compound is an inhibitor of OCT-mediated uptake of platinum drugs in vestibular hair cells is the following:

Organotypic 3D cultures of vestibular explants are incubated with hair cell lethal dose of cisplatin for 48 hrs. Capacities of different OCT inhibitors/substrate to reduce hair cell loss by competing with cisplatin are evaluated using immunohistochemistry and morphometry as quantitative analysis. Comparison used cimetidine competition to assess OCT implication.

Another example of a method for determining if a compound is an inhibitor of OCT-mediated uptake of platinum drugs is described in Kido et al (J Med Chem, 2011, 54(13): 4548-4558). This method is a fluorescence uptake assay, using cell lines stably expressing an OCT receptor and measuring the uptake of ASP+(4-(4-(Dimethylamino)styryl)-N-methyl-pyridinium iodide) using excitation and emission filters at 485 nm and 500-580 nm wavelength respectively. Non-specific transport may be determined using 500 μM cimetidine. After subtracting out the non-specific transport, residual transport rates are used for further calculations. Experimental IC₅₀ are then measured as uptake of ASP+ in the presence of increasing concentrations of a compound. In one embodiment, cells are HEK cells and are seeded in black poly-D-lysine-coated plates at about 45000 cells/well 48 h prior to experiments. Assay buffers may be prepared by diluting 1 mM DMSO stock solution of the compound to be tested with HBSS (pH 7.4) containing ASP+(5 μM) to a final concentration of 20 μM (2% DMSO).

In another embodiment of the invention, said compound is an inhibitor of CTR (copper transporters)-mediated uptake of platinum drugs in the vestibular hair cells and/or vestibular primary neurons.

In one embodiment of the invention, said CTR inhibitor is an inhibitor of CTR1 and/or CTR2, encoded by SLC31A1 and SLC31A2 respectively. Preferably, said CTR inhibitor is a CTR1 inhibitor.

As used herein, the term “inhibitor of CTR-mediated uptake of platinum drugs” refers to any compound that inhibits, impairs or prevents the transport of platinum drugs in vestibular hair cells and/or vestibular primary neurons. For example, said compound may be an inhibitor of the transporter function of the CTR. Another example is a compound that is a substrate of CTR, which means that the compound interacts with the CTR and enters in competition with the platinum drugs, thereby reducing or inhibiting the uptake of platinum drugs within the vestibular hair cells and/or vestibular primary neurons.

Examples of inhibitors of CTR-mediated uptake of platinum drugs in the vestibular hair cells and/or vestibular primary neurons include, but are not limited to, copper sulfate.

Example of a method for determining whether a compound is an inhibitor of CTR-mediated uptake of platinum drugs in the vestibular hair cells is the following:

Organotypic 3D cultures of vestibular explants are incubated with hair cell lethal dose of cisplatin for 48 hrs. Capacities of different CTR inhibitor/substrate to reduce hair cell loss by competing with cisplatin are evaluated using immunohistochemistry and morphometry as quantitative analysis. Comparison used copper sulfate competition to assess CTR implication.

In another embodiment of the invention, said compound is an activator of MATE (multidrug and toxin extrusion) transporters-mediated efflux or excretion of platinum drugs from the vestibular hair cells and/or vestibular primary neurons.

In one embodiment of the invention, said MATE transporters activator is an activator of MATE1, MATE2 and/or MATE2-K, encoded by SLC47A1, SLC47A2 and SLC47A2 respectively. Preferably, said MATE transporter activator is a MATE2 transporter activator. Preferably, said MATE transporter activator is a MATE1 transporter activator.

As used herein, the term “activator of MATE transporter-mediated efflux of platinum drugs” refers to any compound that activates or increases the efflux or excretion of platinum drugs from the hair cells. For example, said compound may be an activator of the transporter function or expression of the MATE transporter.

Examples of activators of MATE transporter-mediated efflux of platinum drugs in the vestibular hair cells include, but are not limited to, drugs activating nuclear receptor transcription factors AhR, CAR, PXR, PPARα and Nrf2 that increase MATE expression.

Example of a method for determining whether a compound is an activator of MATE transporter-mediated efflux of platinum drugs in the vestibular hair cells is the following:

Organotypic 3D cultures of vestibular explants are incubated with hair cell lethal dose of cisplatin for 48 hrs. Capacities of different MATE activators to reduce hair cell loss by enhancing cisplatin extrusion are evaluated using immunohistochemistry and morphometry as quantitative analysis.

In another embodiment of the invention, said compound is an activator of MRP (multidrug resistance-associated protein) transporters-mediated efflux or excretion of platinum drugs in the vestibular hair cells and/or vestibular primary neurons.

In one embodiment of the invention, said MRP transporters activator is an activator of MRP1, MRP2, MRP3, MRP4, MRP5, MRP6, MRP7 and/or MRP8. Preferably, said MRP transporter activator is MRP2 transporter activator. Preferably, said MRP transporter activator is MRP1 transporter activator.

As used herein, the term “activator of MRP transporter-mediated efflux of platinum drugs” refers to any compound that activates or increases the efflux or excretion of platinum drugs from the hair cells. For example, said compound may be an activator of the transporter function of the MRP transporter.

Examples of activators of MRP transporter-mediated efflux of platinum drugs in the vestibular hair cells include, but are not limited to pharmacological agents and endogenous molecules that induce MRP expression via activation of nuclear receptor transcription factors. Examples of said agents or molecules include but are not limited to collagen/β1 integrin, lipopolysaccharide (LPS). Examples of MRP2 activator include, but are not limited to, probenecid, benzylpenicillin, estron-3-sulfate, taurocholic acid, ezrin, glucocorticoids such as cortisol, dexamethasone, triamcinolone acetonide, and RU486. Examples of MRP1 activator include, but are not limited to, epicatechin and interleukins such as IL-6.

Example of a method for determining whether a compound is an activator of MRP transporter-mediated efflux of platinum drugs in the vestibular hair cells is the following:

Organotypic 3D cultures of vestibular explants are incubated with hair cell lethal dose of cisplatin for 48 hrs. Capacities of different MRP activators to reduce hair cell loss by enhancing cisplatin extrusion are evaluated using immunohistochemistry and morphometry as quantitative analysis.

In another embodiment of the invention, said compound is an activator of ATP7A or ATP7B-mediated efflux or excretion of platinum drugs in the vestibular hair cells and/or vestibular primary neurons.

As used herein, the term “activator of ATP7A or ATP7B-mediated efflux of platinum drugs” refers to any compound that activates or increases the efflux or excretion of platinum drugs from the hair cells. For example, said compound may be an activator of the transporter function of the ATP7A or ATP7B transporter.

Examples of activators of ATP7A or ATP7B-mediated efflux of platinum drugs in the vestibular hair cells include, but are not limited to, copper sulfate, 4-phenylbutyrate (4-PBA) and curcumin.

Example of a method for determining whether a compound is an activator of ATP7A or ATP7B-mediated efflux of platinum drugs in the hair cells is the following:

Organotypic 3D cultures of vestibular explants are incubated with lethal dose of cisplatin for 48 hrs. Capacities of different ATP7A or ATP7B activators to reduce hair cell loss by enhancing cisplatin extrusion are evaluated using immunohistochemistry and morphometry as quantitative analysis. Comparison used copper sulfate competition to assess CTR implication.

According to the invention, the compound that impairs accumulation of platinum drugs within vestibular hair cells and/or vestibular primary neurons may be administered in the form of a pharmaceutical composition, as defined below.

Preferably, said compound is administered in a therapeutically effective amount. As used herein, the term “therapeutically effective amount” refers to the level or amount of a compound that is aimed at, without causing significant negative or adverse side effects to the target, (1) delaying or preventing the onset of platinum drugs induced vestibulotoxicity; (2) slowing down or stopping the progression, aggravation, or deterioration of one or more symptoms of platinum drugs induced vestibulotoxicity; (3) bringing about ameliorations of the symptoms of platinum drugs induced vestibulotoxicity; (4) reducing the severity or incidence of platinum drugs induced vestibulotoxicity; or (5) curing platinum drugs induced vestibulotoxicity. A therapeutically effective amount may be administered prior to the onset of platinum drugs induced vestibulotoxicity, for a prophylactic or preventive action. Alternatively or additionally, the therapeutically effective amount may be administered after initiation of platinum drugs induced vestibulotoxicity, for a therapeutic action.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific inhibitor employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 5000 mg per adult per day, preferably from 0.01 to 2500 mg per day, more preferably from 0.01 to 1000 mg per adult per day. Preferably, the compositions contain 0.01, 0.05, 0.1, 0.5, 1.0, 2.5, 5.0, 10.0, 15.0, 25.0, 50.0, 100, 250, 500, 1000, 1500, 2000, 2400, 3000, 3500, 4000, 4500 or 5000 mg of the active ingredient for the symptomatic adjustment of the dosage to the patient to be treated.

The compound may be combined with pharmaceutically acceptable excipients, and optionally sustained-release matrices, such as biodegradable polymers, to form therapeutic compositions.

The term “pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The compound of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active ingredients in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

In a particular embodiment, the compound of the invention may be administered directly in the inner ear through the tympanic membrane. This administration mode may be preferred for introducing a direct and long term effect on the vestibule. Accordingly in a preferred embodiment, the compound of the invention is administered in a gel formulation to allow a long term release of said compound in the inner ear.

In one embodiment of the invention, the compound that impairs accumulation of platinum drugs within vestibular hair cells and/or vestibular primary neurons is administrated concomitantly to the platinum drugs.

In another embodiment of the invention, the compound that impairs accumulation of platinum drugs within vestibular hair cells and/or vestibular primary neurons is administrated before the administration of the platinum drugs.

The invention will be further illustrated by the following figures and examples. However, these examples and figures should not be interpreted in any way as limiting the scope of the present invention.

### BRIEF DESCRIPTION OF THE DRAWINGS

FIG. 1: OCT2 and MATE1 expression and localization.

FIG. 2: OCT1, OCT2, CTR1, CTR2, MATE1, ATP7A, ATP7B expression.

FIG. 3: in vitro impact of cimetidine on cisplatin induced vestibulotoxicity.

FIG. 4: in vivo impact of i.p. administered cimetidine on cisplatin induced vestibulotoxicity.

FIG. 5: in vivo impact of transtympanically administered cimetidine on cisplatin induced vestibulotoxicity.

FIG. 6: in vivo impact of nizatidine on cisplatin induced vestibulotoxicity.

FIG. 7: in vivo impact of ranitidine on cisplatin induced vestibulotoxicity.

FIG. 8: in vivo impact of pantoprazole on cisplatin induced vestibulotoxicity.

FIG. 9: in vivo impact of rabeprazole on cisplatin induced vestibulotoxicity.

FIG. 10: in vivo impact of lansoprazole on cisplatin induced vestibulotoxicity.

FIG. 11: in vivo impact of omeprazole on cisplatin induced vestibulotoxicity.

FIG. 12: ear concentration of compound of the invention 30 minutes or 180 minutes after systemic administration

### EXAMPLES

The present invention is further illustrated by the following examples.

### Material and Methods

### RT-PCR

Adult rats were killed by an overdose of pentobarbital (i.p.) before removal of various organs. Kidney, cochlea and spleen (positive and negative tissue controls) vestibular ganglia and sensory epitheliums were flash frozen in liquid nitrogen immediately after dissection from adult rats. For a single experiment, at least 12 vestibular ganglia and one spleen were used to extract the total RNA using a standard protocol with TRIzol (RiboPure, Ambion, Austin, Tex., USA) and chloroform. First-strand cDNA synthesis (reverse transcription, RT) was performed with 0.5-1 mg of total RNA DNAse treated (Qiagen, Courtaboeuf, France) and Oligo(dT) primers (Kit Quantitect reverse transcription, Qiagen). Reactions were performed at 42° C. for 1 h followed by 95° C. for 2 min. PCR was performed with 1-2 mL of RT reaction material. Amplification was for 35 cycles for spleen and vestibular ganglia with 30 s at 95° C., 30 s at the primer-specific annealing temperature (58-60° C.) and 1 min at 72° C. A final extension was done at 72° C. for 7 min. RT was preferentially done using sequence specific primer and PCR products (1 mL) resolved on 2% agarose gels containing ethidium bromide (10%). Results were obtained in three different experiments (two independent) and PCR products were identified by direct sequencing. To avoid misinterpretation of our results through genomic DNA contaminations, primer sets were designed across intron-exon boundaries as determined from rat sequences using the free NCBI/Primer-Blast software. Primers were systematically tested for quality on spleen tissue. Primers for OCT1: Forward: AAGCACACCGTCATCCTGAT (SEQ ID NO: 1), Reverse: CCAGGTTCTCTGCTTCTTCA (SEQ ID NO: 2); for OCT2: Forward: GCCAGTGCATGAGGTATGAA (SEQ ID NO: 3), Reverse: AGTTGGGAATCACATAGGCC (SEQ ID NO: 4); for MATE1: Forward: ATACGGGAGCCAGAACTTGA (SEQ ID NO: 5), Reverse GTTGACAAGGTTAGCAGCGA (SEQ ID NO: 6); for CTR1: Forward: TCATCATCCTGAGCCTTGTC (SEQ ID NO: 7), Reverse: GACTGGATGAGATGTCCCTA (SEQ ID NO: 8), for CTR2: Forward: GTATGAGGGCATCAAGGTTG (SEQ ID NO: 9), Reverse: CTTCATCTCAGTCACCAGGA (SEQ ID NO: 10); for MRP1: Forward: AACATTGCTACAAGGCGGTG (SEQ ID NO: 11), Reverse: CTTTGACTCCTTCCCTAAAC (SEQ ID NO: 12); for MRP2: Forward: ACTTGGTCGTCTTCTGTTCC (SEQ ID NO: 13), Reverse: GAGGCAACATCTATCCCATC (SEQ ID NO: 14); for ATP7A: Forward: AGCAGCAGATTGGGAAAGTG (SEQ ID NO: 15), Reverse: CAGTGGAGCAGTAACAGTCA (SEQ ID NO: 16); for ATP7B: Forward: TCATCCTGGTGGTTGCCATA (SEQ ID NO: 17), Reverse: CTTGATGATGTCACCTCGCT (SEQ ID NO: 18).

OCT2 Expression. Immunocytochemistry.

Adult animals (12 weeks old, Long Evans rats) were deeply anesthetized with Pentobarbital (0.4%), then perfused transcardially with heparin PBS (0.01 M) followed by a fixative solution (4% paraformaldehyde, 1% picric acid, with 5% sucrose). Temporal bones were postfixed in the same solution before vestibular epithelia were dissected in PBS. Vestibular endorgans were embedded in 4% agarose, and 40 μm thick sections were cut with a vibratome (HM650V, Microm). The free-floating sections were first permeabilized with 4% Triton X-100, non-specific binding was prevented by a pre-incubation step in a blocking solution of 0.5% fish gelatin, 0.5% Triton X-100 and 1% BSA. Samples were then incubated with primary antibodies: monoclonal mouse anti-OCT2 (1:200; Sigma-Aldrich) diluted in the blocking solution. For control experiments, the investigated primary antibody was omitted, while the following procedures were unchanged. Specific labeling was revealed with Alexa 594-fluorescent secondary antibodies (1:700) in the blocking solution combined to actin staining with Alexa 488-conjugated phalloidin (Fisher Scientific, Illkirch, France). Samples were observed with a laser scanning confocal microscope (LSM 5 LIVE DUO, Zeiss). Final image processing was done with Adobe Photoshop software (San Jose, Calif.). Control reactions were observed and processed with the parameters used for the stained sections.

In Vitro. Organotypic 3D Cultures of Rat Utricle.

Vestibular end organs were aseptically removed from animals. Explants composed of sensory and nonsensory epithelia were placed on 5 μl of growth factor-reduced Matrigel on laminin (10 μg/ml)-coated glass coverslips. These preparations were incubated for 30 min at 37° C. in a 95%/5% O₂/CO₂ atmosphere at saturating moisture. Explants were then cultured in DMEM and F12 (50%/50% v/v) media supplemented with N2 mixture (2%). The medium was renewed every 4 d. After 7 d in vitro (7 DIV), utricles were incubated with cisplatin (100 or 1000 μM) with or without cimetidine (1000 μM) for 48 hrs. Treated cultures were then fixed with PAF 4% for 1 hr. Labeling of hair bundles was processed using Alex488-conjugated phalloidin (1:700) overnight. After rinsing with PBS, cultures were mounted in Moviol (Calbiochem). Labelling was observed and image digitalized using an apotome microscope (Zeiss). Hair bundles per utricle were quantified using ImageJ software. Statistical analysis was processed with SigmaPlot software (Systat Software). Final image processing was performed with Adobe Photoshop software.

In Vivo. Induced-Deficits of Vestibular Behavior.

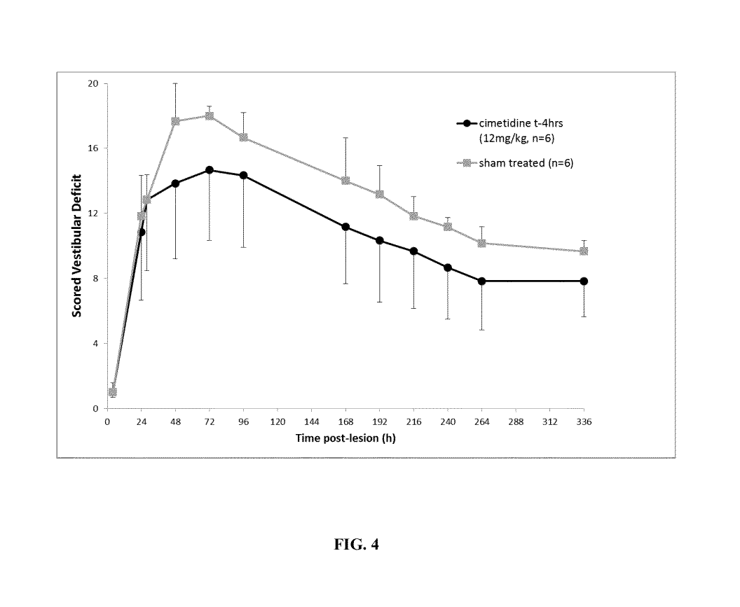

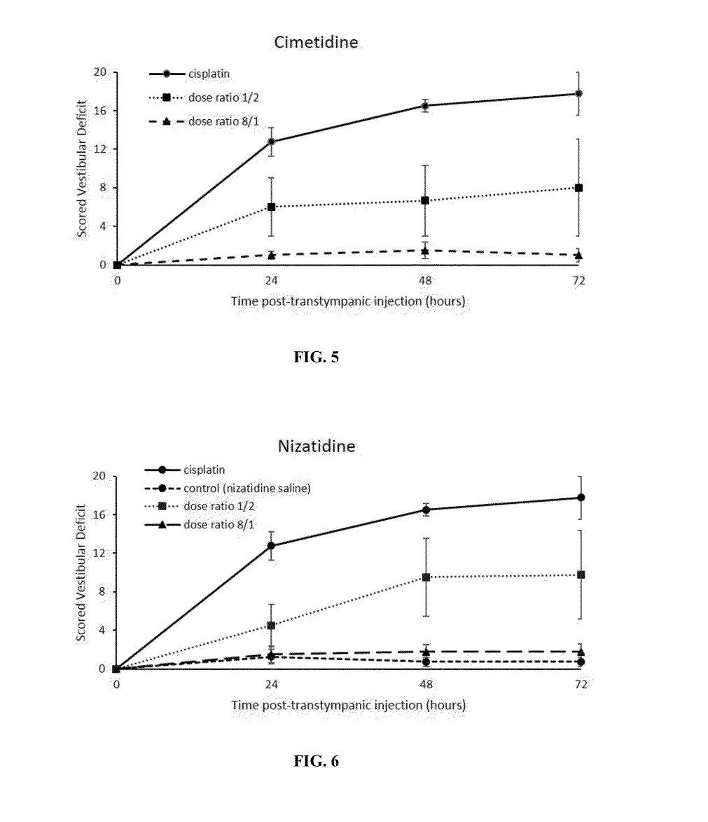

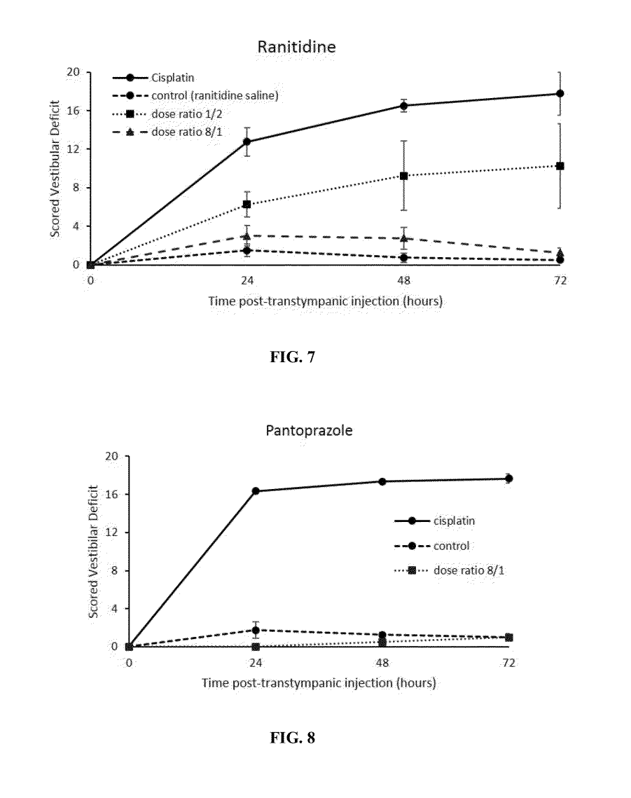

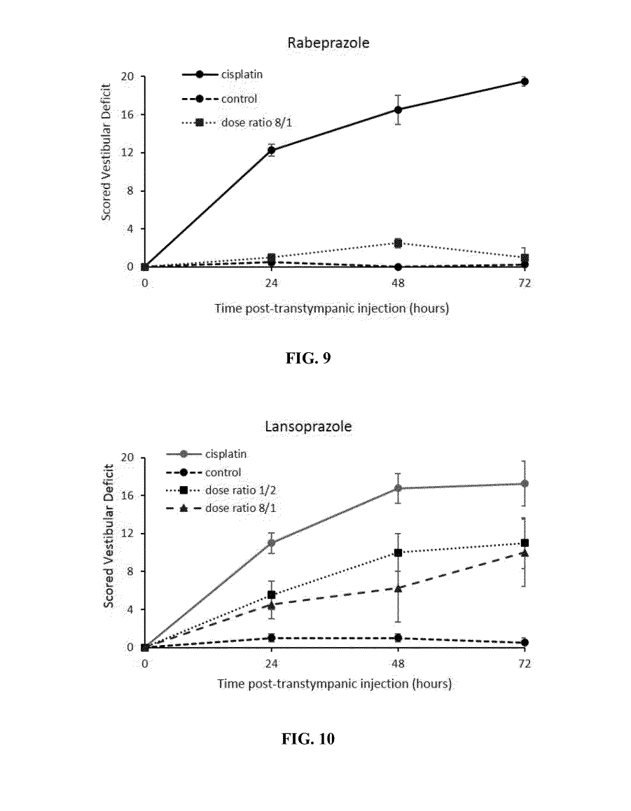

Vestibular deficit associated behaviors were scored on a scale from 0 to 4, respectively ranging from normal behavior (rating 0) to maximal identified vestibular impairment (rating 4). Seven different tests were sequentially scored and totaled to rate the vestibular deficit: 1—head bobbing (abnormal intermittent backward extension of the neck); 2—circling (stereotyped movement ranging from none to compulsive circles around the hips of the animal); 3—retro-pulsion (typical backward walk reflecting vestibular disturbance); 4—tail-hanging reflex (normally induces a forelimb extension to reach the ground, unilateral deficit results in axial rotation of the body); 5—contact-inhibition reflex (normally leads animal to release hold grip on metal grid in a supine position when their back touch the ground; in case of vestibular deficit with lack of full body orientation reference, animal retains grip on the grid in a supine position); 6—air-righting reflex (necessary for rats to land on all four feet when falling from a supine position; vestibular dysfunction impairs normal body repositioning with a maximal deficit leading the animal to land on its back when dropped from a height of 40 cm onto a foam cushion); 7-head tilt. Rats were scored for vestibular deficits after transtympanic injection of cisplatin (50 μl, 2 mg/ml) and, evaluated from 4 to 336 hrs after the injection (FIG. 4). Scores were expressed as mean (±sem). For preventive treatment, injection of cimetidine (i.p., 12 mg/kg) was injected 4 hours before the transtympanic injection of cisplatin. Sham animals received saline (i.p) 4 hours before the lesion. To focus on the preventive action of transtympanic injection of the compounds of the invention in the first 72 hours, ototoxicity is induced by a transtympanic injection (50 μl) of cisplatin (2 mg/ml in saline; 6.7 mM) and a determined ratio of corresponding molar dose of compounds are premixed and injected transtympanically with cisplatin. For example, for the dose ratio 1/2 of compound/cisplatin, a solution of 3.5 mM of compound of interest in addition to the 6.7 mM of cisplatin in saline is prepared and, 50 μl of this mixed saline solution is injected transtympanically at TO. Different control animals are run in parallel: saline only, cisplatin (6.7 mM) only to control the initial lesion, and the compounds of interest alone at different doses in saline to control potential ototoxicity of the compound independently of cisplatin, are with cisplatin. The level of efficacy for the different dose ratio of compounds/cisplatin is compared to 8/1 dose ratio of cimetidine/cisplatin whose efficiency to prevent cisplatin induced ototoxicity is known. For transtympanic experiments, rats were scored for vestibular deficits just before the transtympanic injection, TO and, evaluated at 24, 48 & 72 hours after the injection (FIGS. 5 to 11). Scores were expressed as mean (±sem).

### Inner Ear Exposure of Compounds

In clinical condition, cisplatin is administered via the intravenous route. To evaluate potential efficacy of the compounds of interest to prevent from cisplatin induced ototoxicity following chemotherapy treatment, we evaluate the capacity of each compounds to reach the inner ear compartment. A systemic administration (40 mM; intravenous) of cisplatin or the compound of interest was realized, temporal bones were excised 90 or 180 min after the systemic injection. Inner ear tissue was extracted and the quantity of compounds analyzed by LC/MS appropriate methods. Quantity are expressed as mean (±sem). Four animals of each group were sampled for each time point.

### Results

OCT2 & MATE1 Expression. Immunocytochemistry.

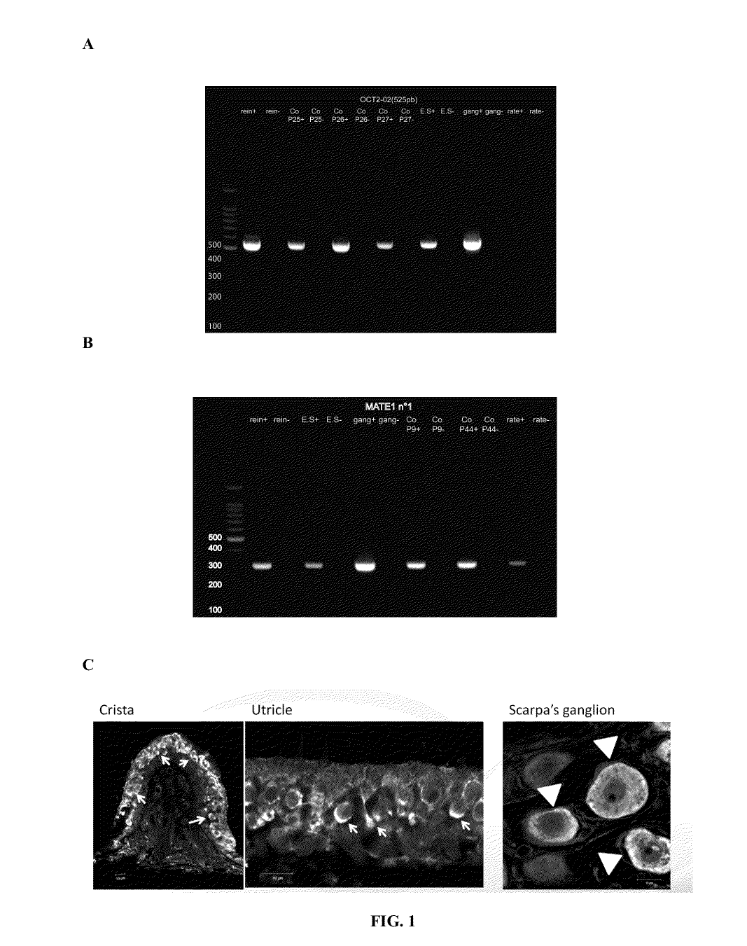

OCT2 & MATE1 mARNs are expressed in the vestibular end organ in both primary ganglions (gang) and sensory epitheliums (E.S.) as compared to positive & negative tissue controls (positive: rein=kidney; Co=cochlea) (negative control: rate=spleen). Sequencing validated the results of RT-PCR (FIG. 1A, B). Expression of OCT2 is specifically revealed by immunohistochemistry and localized in vestibular hair cells (arrows) of both crista and macula and, in primary vestibular neurons (arrowheads) of adult rats (FIG. 1C).

### OCT1, OCT2, CTR1, CTR2, MATE1, MRP1, MRP2, ATP7A and ATP7B Expression.

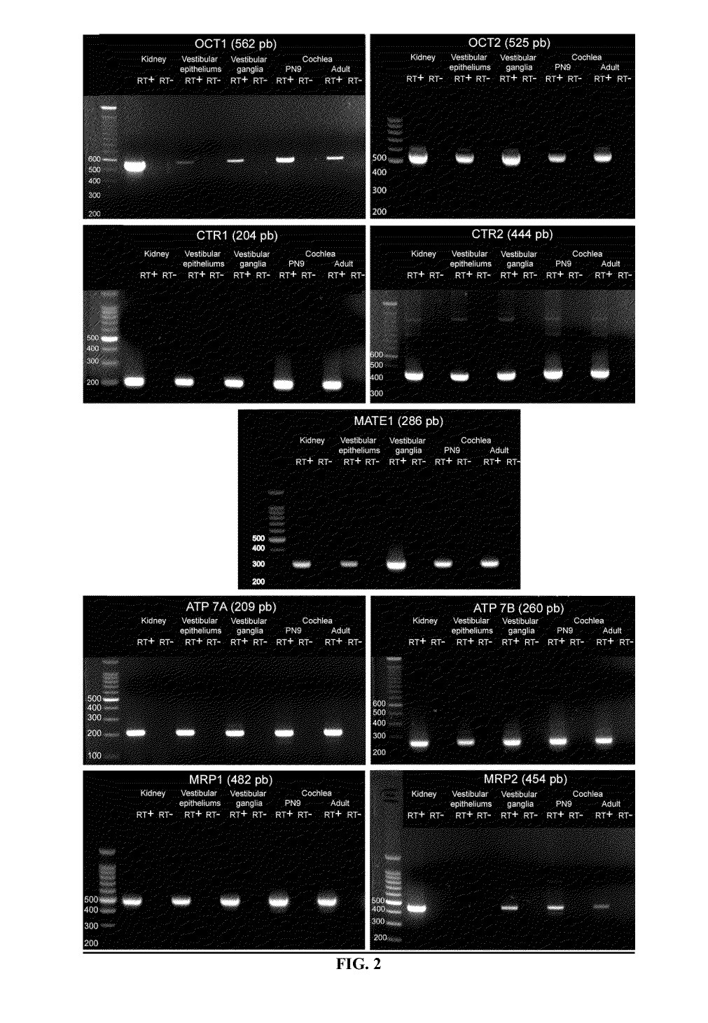

OCT1, OCT2, CTR1, CTR2, MATE1, MRP1, ATP7A and ATP7B mARNs are expressed in the vestibular end organ in both primary ganglions and sensory epitheliums, MRP2, is expressed in primary ganglion as compared to positive (kidney=rein) & negative (without reverse transcriptase=RT-) tissue controls. Sequencing validated the results of RT-PCR (FIG. 2).

### Prevention In Vitro.

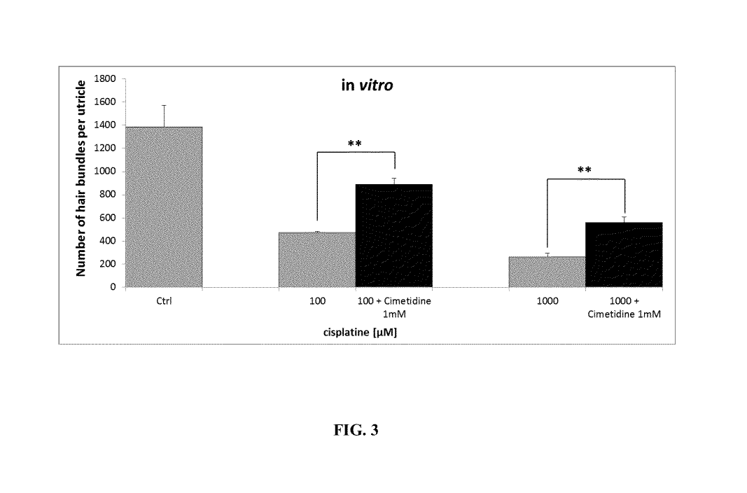

48 hrs incubation of Cisplatin induces vestibular hair cells loss in vitro. Co-treatment with cimetidine reduces vestibular hair cell loss in vitro (FIG. 3).

### Prevention In Vivo.

Pretreatment (t-4 hrs) of adult rats with systemic injection of cimetidine reduces the vestibular toxicity and behavioral deficits induced by cisplatin. Protective effects are observed as soon as 48 hrs after the lesion and persist over the following days (FIG. 4).

Moreover, transtympanic injection of cimetidine, nizatidine, ranitidine, pantoprazole, rabeprazole, lansoprazole and omeprazole also reduces the vestibular toxicity and behavioral deficits induced by cisplatin. Protective effects are observed as soon as 24 hrs after the lesion and persist over the following days (FIGS. 5 to 11).

Ear Concentration of Compounds after Systemic Administration

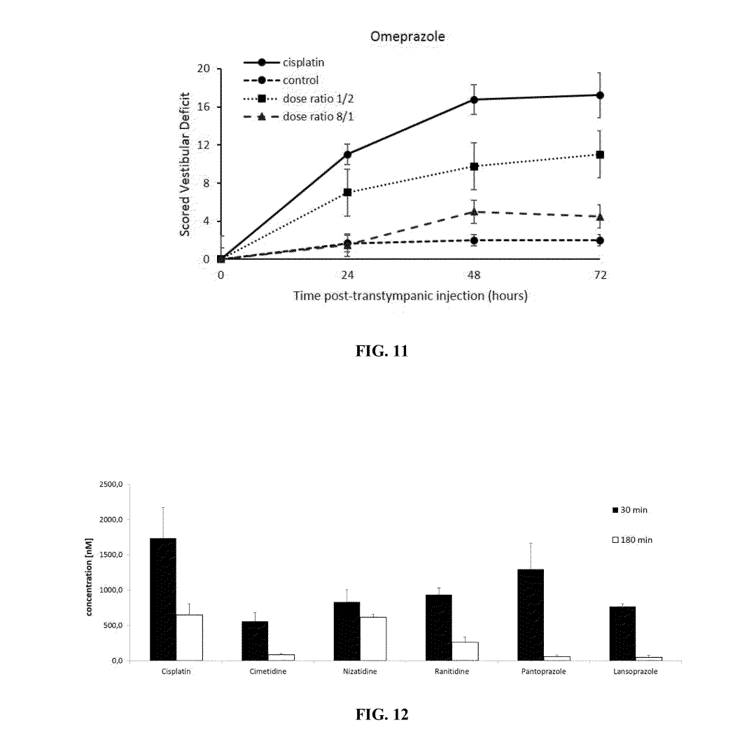

The concentration of the compounds of the invention in the ear 30 minutes after systemic administration reached between 500 and 1500 nM, and the compounds is still detected in the ear 3 hours after the systemic administration (FIG. 12).

These results demonstrated that the systemic administration is an effective way for compound to reach the ear.

1. A method for treating platinum drugs induced vestibulotoxicity, comprising administering a compound that impairs or prevents accumulation of platinum drugs within vestibular hair cells and/or vestibular primary neurons.

2. The method according to claim 1, wherein said compound promote the protection,
survival or regeneration of the vestibular hair cells and/or vestibular primary neurons.

3. The method according to claim 1, wherein the platinum drugs are cisplatin, carboplatin, oxaliplatin, satraplatin, picoplatin, tetraplatin, transplatin, nedaplatin, ormaplatin, PtCl2[R,R-DACH], pyriplatin, ZD0473, BBR3464 or Pt-1C3.

4. The method according to claim 1, wherein said compound modulates platinum drugs flux through vestibular hair cells and/or vestibular primary neurons, or impairs or reduces the influx or uptake of platinum drugs in vestibular cells and/or vestibular primary neurons, or impairs or increases the efflux of platinum drugs in the vestibular hair cells and/or vestibular primary neurons.

5. The method according to claim 1, wherein said compound is an OCT (organic cations transporter) inhibitor, a CTR (copper transporters) inhibitor, a MATE (multidrug and toxin extrusion) transporters activator, a MRP (multidrug resistance associated protein) transporters activator, or an ATP7A or ATP7B transporters activator.

6. The method according to claim 1, wherein said compound is cimetidine, linagliptin, amantadine, memantine, agmatine, chlorpromazine, doxepin, erlotinib, gefitinib, metformin, amitriptyline, carvedilol, chloroquine, clonidine, desloratadine, diphenhydramine, disopyramide, fenfluramine, flecainide, flurazepam, imipramine, ipratropiumbromide, levomethadone, mefloquine, mexiletine, propafenone, propranolol, quinidine, sibutramine, tamoxifen, trimethoprim, verapamil, imatinib, and 6β-hydroxycortisol, collagen/β1 integrin, lipopolysaccharide (LPS), copper sulphate, 4-phenylbutyrate (4-PBA), curcumin, nizatidine, ranitidine, pantoprazole, rabeprazole, lansoprazole or omeprazole.

7. The method according to claim 1, wherein said compound is cimetidine, nizatidine, ranitidine, pantoprazole, rabeprazole, lansoprazole or omeprazole.
